(19) United States
(12) Patent Application Publication
(10) Pub. No.: US 2006/0074045 A1
(43) Pub. Date: Apr. 6, 2006

(54) Vector for transfection of eukaryotic cells

(76) Inventors: Lei Yu, Carlsbad, CA (US); Kenji Matsumoto, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/286,243

(22) Filed: Nov. 23, 2005

Related U.S. Application Data
(63) Continuation of application No. 10/387,213, filed on Mar. 10, 2003.
(60) Provisional application No. 60/363,955, filed on Mar. 12, 2002.

(51) Int. Cl. A61K 48/00; C12N 15/88

(52) U.S. Cl. 514/44; 435/458

(57) ABSTRACT

Vectors comprising a nucleic acid, a nucleic acid binding polymer, a vesicle and a membrane active polypeptide are described. Preferred vectors facilitate transfection and/or reduce cytotoxicity. Methods of making the vectors and methods of using the vectors to transfect cells and/or treat a patient in need of gene therapy are described.

### RELATED APPLICATION INFORMATION

This application is a continuation of U.S. patent application Ser. No. 10/387,213 filed Mar. 10, 2003, which claims priority to U.S. Provisional Application No. 60/363,955, filed Mar. 12, 2002, both of which are hereby incorporated by reference in their entireties.

### FIELD OF THE INVENTION

The present invention relates to a vector comprising a cationic polymer that efficiently condenses nucleic acid and a lipid-based functional vesicle that carries a membrane active agent, such as viral envelope proteins or membrane active peptides which enhance efficiency of transfection of nucleic acids into eukaryotic cells with reduced cytotoxicity. The present invention also relates to methods of transfecting eukaryotic cells with such vectors.

### BACKGROUND OF THE INVENTION

Gene therapy potentially offers a means of treating currently incurable genetic and acquired diseases (Verma, IM. Gene Therapy: Beyond 2000. Mol Ther. 2000 Jun. 1(6):493). However, in this post-genomic era, the main problem with this therapeutic approach is a lack of effective gene delivery systems (Anderson, W F. Human Gene Therapy. Nature 392:25-30 (1998)). Gene delivery systems are designed to protect and control the location of a gene within the body by affecting the distribution and access of a gene expression system to the target cell, and/or recognition by a cell-surface receptor followed by intracellular trafficking and nuclear translocation (Friedmann, T. The Development of Human Gene Therapy. Cold Spring Harbor Laboratory Press. San Diego. 1999). Generally, there are two classes of gene vector systems: viral vector systems and non-viral vector systems. Viral vector systems include retroviral vector systems, lentiviral vector systems, adenoviral vector systems, adeno-associated viral vector systems, HSV viral vector systems, and alpha viral vector systems. Generally, viral vector systems have efficient gene transfer relative to non-viral gene carrier systems, because viruses have developed efficient mechanisms to overcome the gene transfer barrier in human beings. However, viral gene carrier systems have inherent disadvantages for use in the human body, such as the risk of wild-type virus regeneration, high immunogenecity and inflammation, and tumorogenesis (Verma I M, Stevenson J. Gene Therapy—Promises, Problems and Prospects. Nature. 1997 Sep. 18; 389(6648):239-42; Huang, L. and Viroonchatapan, E. Part I: Introduction in Nonviral Vectors for Gene Therapy. In L. Huang, M. C. Hung and E. Wanger (eds.). Nonviral Gene Vectors. Academic Press. (1999)).

The primary concern regarding gene carrier applications in medical gene therapy is safety and the potential of harm to cells. The severe limitations of viral vector systems greatly promote non-viral vector development. Synthetic non-viral gene carrier systems, include either naked plasmid DNA encoding therapeutic protein alone or with gene carriers such as a liposome-based lipoplex system, a polymer-based polyplex system or a lipid-polymer based lipopolyplex system (Felgner, P L., Zelphati, O., and Liang X. Advances in Synthetic Gene-delivery System Technology. In The Development of Human Gene Therapy. Friedmann, T (ed). Cold Spring Harbor Laboratory Press. San Diego. 1999). There are both similarities and difference between lipoplexes and polyplexes. From a physicochemical point of view, in both systems, DNA is incorporated into a complex as a result of bonds between cationic groups of lipids or polycations and anionic groups of the DNA. The driving force for such binding is the release of the low molecular mass counterions associated with the charged lipids or polymers into the external media, which is accompanied by a substantial entropy gain (Radler J O, Koltover I, Salditt T, Safinya C R. Structure of DNA-cationic liposome complexes: DNA intercalation in multilamellar membranes in distinct interhelical packing regimes. Science. 1997 Feb. 7; 275(5301):810-4). In the case of lipoplexes, the self-assembly process requires interaction between lipid molecules, as well as interaction with the DNA itself (Gershon H, Ghirlando R, Guttman S B, Minsky A. Mode of formation and structural features of DNA-cationic liposome complexes used for transfection. Biochemistry. 1993 Jul. 20; 32(28):7143-51). The resulting structure of the lipid molecules in the hydrophobic domain of the lipoplexes is one major factor that determines the macroscopic characteristics of such complexes, and in particular their size, shape, and stability in dispersion. In general, the ability to vary and control these parameters in lipid dispersions is relatively limited. Many lipoplexes are polydisperse and reveal strong non-equilibrium behavior involving variation in size, charge and stoichiometery (Lasic, D D. Liposomes in Gene Delivery, CRC Press, Boca Raton, Fla., USA. 1997; Pouton C W, Lucas P, Thomas B J, Uduehi A N, Milroy D A, Moss S H. Polycation-DNA complexes for gene delivery: a comparison of the biopharmaceutical properties of cationic polypeptides and cationic lipids. J Control Release. 1998 Apr. 30; 53(1-3):289-99; Templeton N S, Lasic D D, Frederik P M, Strey H H, Roberts D D, Pavlakis G N. Improved DNA: liposome complexes for increased systemic delivery and gene expression. Nat Biotechnol. 1997 Jul. 15(7):647-52). In addition, the lipoplex systems are often poorly water soluble and their macroscopic characteristics are unstable over time, limiting their pharmaceutical application.

Cationic polymer systems are similar to the cationic lipid systems in that they also have an overall positive charge and are capable of condensing plasmid DNA via ionic interactions (Kabanov, A V and Kabanov, A V. DNA complexes with polycations for the delivery of genetic material into cells. Bioconjug Chem. 1995 January-February; 6(1):7-20). In contrast to lipid systems, the cationic polymer spontaneously forms complexes with DNA via electrostatic interactions. Self-assembly of polyplexes does not usually require interaction of the polycation molecules with each other. These positively charged DNA particles can efficiently bind to negatively charged cell membranes and thus can enhance DNA uptake by the cells, resulting in enhanced transfection efficiency. In such systems, a degree of flexibility can be achieved by varying the composition of the mixture (Pouton C W, Seymour L W. Key issues in non-viral gene delivery. Adv. Drug Deliv. Rev. 2001 Mar. 1; 46(1-3):187-203). Low immunogenecity typically allows polymers to be a biocompatible material for application in patients.

The cationic polymers commonly used as gene carrier backbones are poly(L-lysine) (PLL), polyethyleneimine (PEI), chitosan, dendrimers, and poly(2-dimethylamino)ethyl methacrylate (pDMAEMA).

Poly-L-lysine (PLL)-based polymers, pioneered in 1987, have been used for gene delivery by employing a targeting ligand, e.g. asialoorosomucoid, for transferring the gene and folate, to facilitate receptor-mediated uptake (Wu, G Y., and Wu, C H. Receptor-mediated in vitro gene transformation by a soluble DNA carrier system. J Biol. Chem. 1987 Apr. 5; 262(10):4429-32; Wu, G Y., and Wu, C H. Receptor-mediated gene delivery and expression in vivo. J Biol. Chem. 1988 Oct. 15; 263(29):14621-4; Mislick K A, Baldeschwieler J D, Kayyem J F, Meade T J. Transfection of folate-polylysine DNA complexes: evidence for lysosomal delivery. Bioconjug Chem. 1995 September-October; 6(5):512-5). PLL/DNA complexes are internalized into cells as a result of the interaction of a ligand displayed at the surface of the complex with the receptor (Wagner E, Zenke M, Cotten M, Beug H, Birnstiel M L. Transferrin-polycation conjugates as carriers for DNA uptake into cells. Proc Natl Acad Sci USA. 1990 May; 87(9):3410-4). PLL-mediated gene transfer efficiency has been modified by employing lysosomaotropic agents (such as chloroquine) or inactivated adenovirus, or peptide derived from Haemophilus Influenza envelop proteins to facilitate PLL/DNA complex release from the endosomes (Wagner E, Plank C, Zatloukal K, Cotten M, Birnstiel M L. Influenza virus hemagglutinin HA-2 N-terminal fusogenic peptides augment gene transfer by transferrin-polylysine-DNA complexes: toward a synthetic virus-like gene-transfer vehicle. Proc Natl Acad Sci USA. 1992 Sep. 1; 89(17):7934-8; Curiel D T, Wagner E, Cotten M, Birnstiel M L, Agarwal S, Li C M, Loechel S, Hu P C. High-efficiency gene transfer mediated by adenovirus coupled to DNA-polylysine complexes. Hum Gene Ther. 1992 April; 3(2):147-54). Without the use of either targeting ligands or endosome lytic reagents, gene transfer is typically poor with PLL polyplexes alone, an important difference between the biological activity of the amphiphilic cationic lipids and the soluble polymer PLL.

Unlike PLL, both branched and linear polyethylenimine (PEI) show efficient gene transfer without the need for endosomlytic or targeting agents (Boussif O, Lezoualc'h F, Zanta M A, Mergny M D, Scherman D, Demeneix B, Behr J P. A versatile vector for gene and oligonucleotide transfer into cells in culture and in vivo: polyethylenimine. Proc Natl Acad Sci USA. 1995 Aug. 1; 92(16):7297-301). Postively charged PEI polyplexes are endocytosed by cells, and PEI is also believed to facilitate endosomal escape. Unfortunately, PEI has also been reported to be toxic to cells, which severely limits the potential for using PEI as a gene delivery tool in applications to human patients.

A range of polyamidoamine (PAMAM) dendrimers have been studied as gene-delivery systems (Eichman J D, Bielinska A U, Kukowska-Latallo J F, Baker J R Jr. The use of PAMAM dendrimers in the efficient transfer of genetic material into cells. Pharm. Sci. Technol. Today. 2000 July; 3(7):232-245). Terminal amino groups bind DNA electrostatically, forming positively charged complexes, which are taken up by endocytosis. There are advantages associated with the star shape of the polymer as DNA appears to interact primarily with the surface primary amines, leaving the internal tertiary amines available to assist endosomal escape of the dendrimer-gene complex. Unfortunately, dendrimers have also been reported to be toxic to cells, a major limitation for its application in human patients.

Transfection efficiency and cytotoxicity are two of the most important factors that determine the yield of gene expression. All current cationic polymer gene delivery systems have drawbacks that hinder their use in gene therapies. The first drawback is that these systems are generally much less efficient in gene transfer experiments compared with viral systems, especially in the case of PLL. The second drawback is that the cationic polymer gene carrier systems with higher gene transfer efficiency relative to PLL are usually toxic to the cells.

The natural process of viral infection is the transduction of foreign nucleic acids, the viral genome, into host cells. The cytoplasmic membrane, endosome membrane and nuclear membrane are the three major intracellular barriers of virus transduction. To obtain high infectivity, some viruses have developed an envelope surrounding the virus, where envelope proteins are integrated in the envelope. Usually, viral envelope proteins have two functions: receptor binding and membrane fusion. Receptor binding facilitates transport of the virus through the cell wall via receptor mediated endocytosis, and membrane fusion facilitates the escape of the virus from the endosome/lysosome, resulting in an increase in the number of transfected polymer/gene complexes transported into the nucleus.

Incorporation of viral elements into polymeric gene carrier systems is a strategy to enhance cationic gene carrier mediated gene transfer efficiency and reduce cytotoxicity. Several viruses or viral envelope components have been used to modify lipid-mediated gene transfer in vitro and in vivo. UV inactivated whole defective Sendai virus (hemagluttinating virus of Japan, HVJ) has been used in lipid-based gene carrier systems to improve gene transfer in vitro and in vivo (Saeki Y, Matsumoto N, Nakano Y, Mori M, Awai K, Kaneda Y. Development and characterization of cationic liposomes conjugated with HVJ (Sendai virus): reciprocal effect of cationic lipid for in vitro and in vivo gene transfer. Hum Gene Ther. 1997 Nov. 20; 8(17):2133-41). Curiel, D. T. et al. have reported that receptor-mediated transfection via transferrin-polylysine/DNA complexes is enhanced by simultaneously exposing the cells to defective adenovirus particles (Curiel D T, Agarwal S, Wagner E, Cotten M. Adenovirus enhancement of transferrin-polylysine-mediated gene delivery. Proc Natl Acad Sci USA. 1991 Oct. 1; 88(19):8850-4). These authors report that adenovirus particles function to disrupt endosomes containing the viral particle and the DNA complex. Replication-defective adenovirus particles and psoralen-inactivated adenovirus were reported to enhance transfection. Adenovirus enhancement of transfection is limited, however, to cells which have both a ligand receptor, e.g., transferrin receptor, and an adenovirus receptor. Direct coupling of polylysine/DNA complexes to adenoviruses has also been employed for transfection (Curiel D T, Wagner E, Cotten M, Birnstiel M L, Agarwal S, Li C M, Loechel S, Hu P C. High-efficiency gene transfer mediated by adenovirus coupled to DNA-polylysine complexes. Hum Gene Ther. 1992 April; 3(2):147-54). Even though effective, the whole virus employed in the gene transfection system carries the risks of wild-type virus reformation and viral genome contamination. In related work, Wagner, E. et al. report augmentation of transfection in several cell lines when hemagglutinin HA-2 N-terminal fusogenic peptides from influenza virus are included in transferrin-polylysine-DNA complexes (Wagner E, Plank C, Zatloukal K, Cotten M, Birnstiel M L. Influenza virus hemagglutinin HA-2 N-terminal fusogenic peptides augment gene transfer by transferrin-polylysine-DNA complexes: toward a synthetic virus-like gene-transfer vehicle. Proc Natl Acad Sci USA. 1992 Sep. 1; 89(17):7934-8). Vesicular stomatitis virus G envelope protein (VSVG) has also been reported to help lipid-DNA complex in vitro gene transfer (Abe A, Chen S T, Miyanohara A, Friedmann T. In vitro cell-free conversion of noninfectious Moloney retrovirus particles to an infectious form by the addition of the vesicular stomatitis virus surrogate envelope G protein. J. Virol. 1998 August; 72(8):6356-61; Abe A, Miyanohara A, Friedmann T Enhanced gene transfer with fusogenic liposomes containing vesicular stomatitis virus G glycoprotein. J. Virol. 1998 July; 72(7):6159-63).

The glycoprotein (VSVG) derived from the vesicular stomatitis virus, a member of the rhabdovirus family, is a viral envelope protein that has been widely used in pseudotyping viral vectors to improve gene transduction efficiency. VSVG is a transmembrane protein and induces membrane fusion at acidic pH in the absence of other viral components (Florkiewicz R Z, Rose J K A cell line expressing vesicular stomatitis virus glycoprotein fuses at low pH. Science 1984 Aug. 17; 225(4663):721-3; Riedel H, Kondor-Koch C, Garoff H Cell surface expression of fusogenic vesicular stomatitis virus G protein from cloned cDNA. EMBO J. 1984 July; 3(7):1477-83). Exposure of G protein to acidic pH induces a conformational change which allows the protein to interact simultaneously with the receptor and the target membrane, presumably via hydrophobic amino acids, to induce the membrane fusion (White J M. Membrane fusion. Science 1992 Nov. 6; 258(5084):917-24; White J M. Viral and cellular membrane fusion proteins. Annu. Rev. Physiol. 1990; 52:675-97; Stegmann T, Doms R W, Helenius A Protein-mediated membrane fusion. Annu. Rev. Biophys. Biophys. Chem. 1989; 18:187-211; Puri A, Winick J, Lowy R J, Covell D, Eidelman 0, Walter A, Blumenthal R Activation of vesicular stomatitis virus fusion with cells by pretreatment at low pH. J. Biol. Chem. 1988 Apr. 5; 263(10):4749-53). It has been reported that incorporation of VSVG into liposome enhanced liposome-mediated gene transfection by 7-fold in vitro (Abe A, Chen S T, Miyanohara A, Friedmann T. In vitro cell-free conversion of noninfectious Moloney retrovirus particles to an infectious form by the addition of the vesicular stomatitis virus surrogate envelope G protein. J. Virol. 1998 August; 72(8):6356-61). However, to our knowledge there are no reports that VSVG enhanced cationic polymer based gene carrier system gene transfer efficiency and reduced the cytotoxicity.

### SUMMARY OF THE INVENTION

In preferred embodiments, the present invention provides vectors and methods for transfecting eukaryotic cells, particularly higher eukaryotic cells, with nucleic acids. Nucleic acids, both DNA and RNA, linear or circular, are preferably introduced into cells such that they retain their biological function. The vector for transfecting eukaryotic cells preferably comprises a nucleic acid, a nucleic acid binding polymer, a lipid-based vesicle, and a membrane active polypeptide, such as a viral envelope protein or a peptide derived from the envelope protein which retains functions of the viral envelope protein. Preferred vectors have significantly improved transfection efficiency and cytotoxicity as compared to similar vectors lacking a lipid-based vesicle and a membrane active polypeptide.

The nucleic acid may be deoxyribonucleic acid (DNA), ribonucleic acid (RNA) or a DNA/RNA hybrid and may be in the form of a linear molecule or a circular molecule, such as a plasmid. The nucleic acid may be a single stranded oligodeoxynucleotide. An RNA may be a single or double-stranded RNA and may be a small interference RNA (siRNA) or a ribozyme.

Preferred transfection vectors contain nucleic acid binding polymers which self-assemble in a complex with nucleic acids. These polymers effectively condense the nucleic acid and facilitate introduction of anionic macromolecules, like nucleic acids, through cell membranes, which are typically negatively charged. Preferred types of nucleic acid binding polymers include polymers that are linear, branched, star-shaped, grafted co-polymers, block copolymers, and dendrimers. A dendrimer may have more than three branches. The nucleic acid binding polymer may have a molecular weight of 400 daltons (Da) or more. The polymers may be biodegradable or non-biodegradable. Preferred examples of biodegradable polymers include hydrolysable polymers, pH sensitive polymers, light sensitive cleavable polymers, temperature sensitive cleavable polymers, sonication sensitive cleavable polymers, and enzymatically cleavable polymers. In some embodiments, the polymers are cationic polymers. Some examples of cationic polymers that may be used in preferred embodiments include poly-L-lysine (PLL), polyethelenimine (PEI), poly[a-(-aminoutyl)-L-glycolic acid] (PAGA), chitosan, polyamidoamine (PAMAM), and poly(2-dimethylamino)ethyl methacrylate (pDMAEMA). Preferred complexes of nucleic acid and cationic polymer may contain various amounts of both components. Some embodiments of the invention have a ratio between cationic polymer and nucleic acid in the range of about 1:1 to 50:1, by weight.

Preferred transfection vectors comprise a vesicle having a native or synthetic lipid bilayer and a membrane active polypeptide that functions to facilitate entry of cationic polymer/nucleic acid complexes into a cell. In preferred embodiments, the native or synthetic phospholipid bilayer provides the microenvironment for fusion of the vector with the cell membrane. Various lipid mixtures may be used. One embodiment comprises a mixture of phosphatidylcholine (PC), phosphatidylethanolamine (PE) and phosphatidylserine (PS). In a preferred embodiment, these three lipid components are in a ratio of about 6:2:2, respectively, by weight. Examples of membrane active polypeptides useful in transfection vectors are wild type viral envelope proteins and recombinant envelope proteins. Examples of viral protein include viral envelope vesicles, viral spike glycoproteins, multimers (e.g., dimers, trimers, or oligomers) thereof, peptides of viral spike glycoproteins, and envelope fragments containing embedded viral protein. In a preferred embodiment, vesicular stomatitus virus envelope protein, known as vesicular stomatitus virus glycoprotein (VSVG), is used. VSVG may be a multimer form, with multiple molecules of the protein together in a functional unit, or monomers of the protein, or a combination of both multimers and monomers; the VSVG may be wild type VSVG mature protein, a wild type VSVG peptide or a recombinant VSVG polypeptide. In some embodiments, transfection vectors comprising viral components of any enveloped viruses may be used. Other embodiments may have a non-viral protein as the membrane active polypeptide.

In preferred embodiments, the nucleic acid binding polymer and the nucleic acid form a complex. Such complexes may be formed spontaneously by adding the nucleic acid binding polymer to a nucleic acid solution at the ratio desired in the final product. After the polymer has condensed the nucleic acid to form a complex, the complex is preferably combined with a lipid-based vesicle incorporating a membrane active polypeptide, preferably resulting in containment of the complex within the vesicle or binding of the complex by the vesicle. Accordingly, in one embodiment the complex of nucleic acid binding polymer and nucleic acid is encapsulated in a lipid-based vesicle with a membrane active polypeptide. In another embodiment, the complex of nucleic acid binding polymer and nucleic acid is bound to a lipid-based vesicle with a membrane active polypeptide.

Another embodiment provides a method for making a vector for transfecting a eukaryotic cell by isolating a nucleic acid, combining it with a nucleic acid binding polymer to form a complex, and combining the complex of nucleic acid and polymer with a solution containing lipid-based vesicles, where at least a portion of those vesicles has a membrane active polypeptide in contact with the vesicle. Some embodiments of this method use a cationic polymer as the nucleic acid binding polymer; in particular embodiments, a cationic polymer for use with this method may be poly-L-lysine, polyethylenimine, poly[a-(-aminoutyl)-L-glycolic acid], chitosan, polyamidoamine, or poly(2-dimethylamino)ethyl methacrylate. Some embodiments use a biodegradable polymer in the making of a vector. Examples of preferred biodegradable polymers include hydrolysable polymers and pH-sensitive cleavable polymer. The hydrolysable polymer may be a biodegradable cationic polymer. The pH-sensitive cleavage polymer may be a polyacetal polymer. In preferred embodiments, lipid-based vesicles may contain native lipid membrane or synthetic lipid membrane. In some embodiments of the invention, the membrane active polypeptide is VSVG; in other embodiments, the membrane active polypeptide is a part of VSVG that retains activity with regard to membrane fusion.

Other embodiments include a method of gene therapy where an individual in need of gene therapy is identified and is administered a vector in a therapeutically effective amount. In preferred embodiments, the individual is a mammal.

Additional embodiments include a method of introducing a nucleic acid into a cell by contacting the cell with a vector as described herein. The cell is preferably a eukaryotic cell. Examples of eukaryotic cells that may be used include a human fibroblast, an animal embryonic stem cell, a keratinocyte, a pancreatic cell, a myocardium cell, a bone marrow cell, a neuronal cell, and a macrophage.

These and other embodiments are described in greater detail below.

### BRIEF DESCRIPTION OF THE DRAWINGS

FIG. 1 is an illustration of the preparation of a functional vesicle, specifically a vesicular stomatitis virus glycoprotein (VSVG) vesicle.

FIG. 2 is an image of a Western blot which resulted in the detection of VSVG from both cell lysate and cell conditioned medium preparations as described in Example 1.

FIG. 3 shows the effects of VSVG vesicles on cationic polymer mediated gene delivery in HT1080 cells via β-galactosidase gene staining (for visualization) (3A and 3B) and in HepG2 cells (hepatic carcinoma cells) via luciferase activity measurement (quantification) (3C).

FIG. 4 shows reproductions of FITC assays illustrating the effects of VSVG vesicles on cationic polymer mediated antisense oligonucleotide delivery in 293 cells. FIGS. 4A and 4B show antisense oligonucleotide delivery mediated by cationic polymer PEI or PLL alone. FIGS. 4C and 4D shows delivery by cationic polymer PEI or PLL with VSVG vesicle (250 ng). The antisense oligonucleotide was labeled with FITC fluorescent tag.

FIG. 5 is a diagram illustrating a synthetic method for making biodegradable polymers.

### DETAILED DESCRIPTION OF THE PREFERRED EMBODIMENTS

It has now been discovered that viral envelope components may be used to significantly enhance the efficiency of cationic polymer-mediated transfection of eukaryotic cells. In contrast to previous systems, the enhanced transfection systems described herein preferably include four major components: a nucleic acid binding polymer, a nucleic acid molecule, a native or synthetic lipid bilayer and a membrane active polypeptide. This invention is not bound by theory, but it is believed that the nucleic acid binding polymer interacts with nucleic acid molecules to spontaneously form a complex via ionic interactions. Condensing nucleic acid via the interaction with the nucleic acid binding polymer before introduction of the nucleic acid to a lipid component is believed to overcome the drawback of cationic lipid-based gene carrier systems that exhibit poor condensation efficiency. In preferred embodiments, condensed DNA/polymer complexes are encapsulated in native or synthetic lipid bilayer vesicles containing membrane active polypeptides, also referred to herein as functional vesicles. It is believed that the native or synthetic phospholipid bilayer provides a microenvironment for vector fusion with a cell membrane. Membrane active polypeptides embedded in the lipid bilayer are believed to enhance the gene carrier system's ability to escape from endosomes, resulting in enhanced transfection efficiency. It is believed that the containment of the polymer/DNA complex within functional vesicles produces the reduced toxicity observed in preferred embodiments, as compared to other transfection systems. In addition, in those embodiments where functional vesicles are associated with VSVG or a membrane active fragment of VSVG, preferred methods of transfection may be applicable to a wider range of cell-types than previous techniques because the phospholipid receptor of VSVG is present on many cell surfaces. Preferably, specific ligand receptors in target cell lines are not required. In some embodiments, the VSVG present within the vector is in the form of a single polypeptide, which preferably enhances the stability of the vector. In preferred embodiments, the addition of VSVG vesicles to host cells enhances polymer-based gene transfection and reduces the cytotoxicity of nucleic acid binding polymers to the host cells in vitro, as compared to other systems.

Preferred embodiments provide improved methods for nucleic acid binding polymer-based gene transfer of nucleic acids and other biomolecules to eukaryotic cells. This invention is not bound by theory, but it is believed that this improvement results from the use of a functional vesicle having a native or synthetic lipid bilayer and a membrane active agent, to enhance the efficiency of transfection, to broaden the range of types of cells that can be transfected, to reduce cytotoxicity, or a combination of those effects. Preferred embodiments have significant advantages over methods used in previous transfection systems that employ viruses. With preferred embodiments, there is no limit on the size or composition of nucleic acid that may be transfected, no requirement for chemical modification of the nucleic acid, and no risk of virus contamination in the vector preparation, wild type virus regeneration, or tumorgenesis.

Preferred embodiments have significant advantages over viral-lipid systems. In preferred embodiments, nucleic acid binding polymer-based gene carriers may self-assemble without requiring additional reagents, in contrast to some previous transfection systems, such as a lipid gene carrier system. Preferred vectors are water soluble, stable and do not trigger an immune response. The risk of active virus contamination or wild-type virus regeneration in other systems created by the use of whole virus as a reagent for enhancing transfection is avoided in preferred embodiments because no viruses or replication-competent viral genetic material are used in the preparation of the vectors.

Preferred embodiments comprise a lipid bilayer which is capable of maintaining the vector as an integrated whole. VSVG, for example, may be held in the bilayer and thus resistant to detachment from the vector by shearing forces which may be encountered when the vector is administered in vivo. Preferred embodiments of the vectors described herein have a lipid bilayer comprising a membrane active protein or peptide. In these embodiments, the bilayer maintains the vector as an integrated whole and prevents disassociation of the membrane active portion from the vector.

Preferred embodiments also have advantages over previous transfection methods mediated by lipid-based gene delivery systems. The pretreatment of nucleic acid with cationic polymer before the introduction of the lipid vesicle in preferred embodiments overcomes the drawback of poor nucleic acid condensation by the lipid used in previous transfection methods, resulting in enhanced transfection efficiencies.

Preferred embodiments also have advantages over previous transfection methods wherein the gene carrier systems employ cationic polymer alone. In preferred embodiments, inclusion of a functional vesicle with membrane active polypeptide, protein or derived peptides in a transfection vector comprising nucleic acid binding polymer in a complex with nucleic acid significantly enhances transfection compared to the transfection mediated by the nucleic acid binding polymer with nucleic acid alone. For example, addition of VSVG envelope vesicles to a complex of PLL/DNA was found to enhance gene transfer by up to 1000 times. Incorporation of VSVG vesicle to PEI/pDNA complexes was found to enhance gene transfer efficiency in HT1080 human fibrosarcoma cells by up to 8 to 10 times. Inclusion of VSVG vesicles was found to increase the rates of gene transfer by commercially available dendrimer polymer-mediated gene transfer systems by 7 times. Enhancement of transfection by VSVG vesicles was also demonstrated in primary cell lines that have been found to be difficult to transfect by known cationic polymer-mediated transfection methods. Enhancement of transfection by functional vesicles with virally derived membrane active polypeptides may occur in any cells that the virus can enter and infect in its normal life cycle, without risk of virus contamination. Enhancement of transfection by vesicular stomatitis virus may occur with a wide range of cells, particularly cells which contain “rich receptors”, such as acidic phospholipid phosphatidylserine.

Preferred embodiments do not require the use of a ligand that binds to receptors on the surface of target cells and nucleic acid-complexing agents need not be chemically linked to such ligands nor to functional vesicles. Such embodiments are useful for transfection of a wider range of cell types, including types that are difficult to transfect with previous transfection methods. Preferred embodiments employ, for instance, envelope vesicles of rhabdovirus, preferably vesicular stomatius virus envelope vesicles, that are functional with a wide range of higher eukaryotic cells, both from vertebrates and invertebrates, including mammalian, avian, reptilian, amphibian and insect cells. Preferred embodiments that employ, for example, envelope vesicles of rhabdovirus, preferably vesicular stomatius virus envelope vesicles, significantly reduce the cytotoxicity associated with cationic polymer gene carrier-mediated gene transfection. This reduction in cytotoxicity is believed to be due to receptor-mediated endocytosis of viral envelope, which assists in the entry of the vector contents into the cell, and the protection of the cationic polymers by the lipid bylayers of the vector.

As used herein, the term “nucleic acid binding polymer” means an organic polymer that interacts with a nucleic acid to form a physical association with the nucleic acid. Such interaction may be, for example, an attraction due to charge differences between the polymer and the nucleic acid. Nucleic acid binding polymers are preferably cationic polymers. Preferred cationic polymers include linear and branched polymers, biodegradable and non-biodegradable polymers, polymers with or without conjugation with other functional groups, and with or without lipid incorporation or conjugation. Preferred embodiments include cationic polymers that are capable of forming complexes with nucleic acid.

The term “transfection” is used herein generally to mean the delivery and introduction of biologically functional nucleic acid into a cell, e.g., a eukaryotic cell, in such a way that the nucleic acid retains its function within the cell. Transfection encompasses delivery and introduction of expressible nucleic acid into a cell such that the cell is rendered capable of expressing that nucleic acid. The term “expression” means any manifestation of the functional presence of the nucleic acid within a cell, including both transient expression and stable expression. The term “nucleic acid” encompasses both DNA and RNA without size limits from any source comprising natural and non-natural bases. Nucleic acids may have a variety of biological functions. They may encode proteins, comprise regulatory regions, function as inhibitors of gene or RNA expression (e.g., antisense DNA or RNA or RNAi), function as inhibitors of proteins, function to inhibit cell growth or kill cells, catalyze reactions, or function in a diagnostic or other analytical assay. Nucleic acids used in preferred embodiments may be in a variety of forms. They may be single stranded, double stranded, branched or modified by the ligation of non-nucleic acid molecules. They may be in a linear form or a closed circle form. In some embodiments, plasmid DNA is used as the nucleic acid. Plasmid DNA is a variety of closed circular DNA and preferably contains a bacterial origin of replication or an equivalent sequence that allows the replication of the DNA molecule in a biological system.

The term “lipid-based vesicle” means a small (subcellular) container having walls made up of lipids. The lipids may be arranged in monolayers or bilayers. Preferred embodiments include lipid based vesicles where a lipid bilayer covers, contacts or encapsulates a complex of polymer and nucleic acid. In some embodiments, lipid based vesicles contain other molecules in addition to the lipid molecules. For example, lipid based vesicles may contain membrane active polypeptides in some embodiments. In particular embodiments, the membrane active polypeptides associated with lipid-based vesicles are viral envelope proteins, such as VSVG. A membrane active polypeptide or agent is a polypeptide or other biomolecule in a lipid-based vesicle that interacts with a cellular membrane to increase the likelihood of fusion of the lipid-based vesicle with the cellular membrane being contacted. The lipid-based vesicle containing such a membrane active polypeptide or agent may be referred to herein as a functional vesicle.

The term “cytotoxicity” refers to the loss of cell viability after cell exposure to a component or a solution of a gene delivery system. In preferred embodiments, transfection methods employ cationic polymers in combination with vesicular stomatitis virus envelope vesicles composed of VSVG protein and lipid bilayer cell membrane. The methods of these embodiments have been shown to significantly enhance transfection (e.g., 1000 fold compared to PLL alone) over previous transfection methods that employ comparable cationic polymers without a lipid vesicle.

Functional vesicles are believed to facilitate the entry into a cell of a gene carrier, such as a cationic polymer/DNA complex, and/or the release of gene carriers from compartments or organelles within the transfected cells. In some embodiments, a membrane active polypeptide serves to promote the transport of complexes of polymer and nucleic acid into endosomal or other compartments within the cell being transfected. In some embodiments, membrane active agents include proteins, peptides and other molecules which may facilitate fusion of a gene carrier to a cell membrane and/or the penetration of a cell membrane to facilitate transport of the gene carrier into the cell. Preferably the peptides or biomolecules on the surface of the vesicles are derived from viral envelope protein, but may also include non-viral proteins or peptides that have equivalent or similar functions as viral envelope proteins.

Transfection activity or efficiency may be measured by detecting the presence of the transfected nucleic acid in a cell. Preferably the presence of a transfected nucleic acid is detected by measuring the biological function of the peptide encoded by the nucleic acid in the cell. More preferably, it is assessed by measuring the level of transient or stable expression of a reporter gene contained in the transfected nucleic acid. The level of reporter gene expression may depend on, among other things, the amount of nucleic acid transfected and on the level of activity of the reporter gene's promoter in the type of cell being transfected. Generally, there are two classes of reporter gene detection systems used for reporter gene assays to determine gene transfer efficiency: quantitation and visualization. Quantitative methods use the appropriate substrates to measure a reporter gene product's activity. For example, the bioluminescent enzyme luciferase catalyzes the oxidative carboxylation of beet luciferin, emitting photons that may be measured using a luminometer. The amount of luciferase activity is usually proportional to the overall efficiency of transfection for a cell sample. In one common approach to measuring luciferase activity in a sample of transfected cells, cell extracts are prepared and the amount of luciferase activity in the extract is determined. Measurements of the activities of reporter gene products may be used in turn to determine the gene transfection efficiency. Transfection activity may also be assessed by determining the percent of cells in a sample that have been transfected. With these techniques, individual cells are visualized under a microscope and the number of cells exhibiting characteristics of the transfected reporter gene are counted. For example, cells transfected with the reporter gene β-galactosidase undergo X-gal staining, during which the β-galactosidase present in a cell will hydrolyze X-gal (5-bromo-4chloro-3-indoyl-β-D-galactopyranoside) and yield a blue precipitate. Other detection and quantitative methods which may be used are well known in the art.

Preferred embodiments include methods useful for the transfection of cells that have commonly been difficult to transfect by previous techniques. These previous techniques include those that use cationic polymers, such as poly-L-lysine. The term “difficult to transfect” refers to those eukaryotic cell lines in which, under transfection assay conditions as described in Example 3, less than about 1% of the cells in a sample are transfected employing the cationic polymer reagent poly-L-lysine alone. “Difficult to transfect” cells include animal primary cell lines such as human fibroblasts, animal embryo stem line cells, keratinocytes and macrophages.

Preferred embodiments provide methods comprising contacting a eukaryotic cell with a transfection vector comprising a cationic polymer, a functional vesicle containing native or synthetic lipid bilayer, and a nucleic acid or other biomolecule. The functional vesicle may comprise an envelope of a vesicular stomatitis virus, an alphavirus, or an influenza virus or a component thereof. Enhanced transfection methods of these embodiments have been demonstrated with the prototype envelope vesicle from vesicular stomatitis virus envelope vesicle (VSV-G vesicle) and the prototype vesicular stomatitis virus G protein (VSV-G protein). VSVG has three domains: cytoplasmic, transmembrane, and extracellular. The extracellular domain is the fusogenic portion of the protein and has the functions of recognizing a receptor or target on the surface of a cell, fusing to the cell and/or penetration of the cell membrane.

In preferred embodiments, a cationic polymer forms a cationic polymer/nucleic acid complex. Preferably, the cationic polymer spontaneously form a complex with nucleic acid in aqueous solution. Various well-known techniques may be employed to produce a desired type of cationic polymer/nucleic acid complex. The relative amounts of cationic polymer employed to form the complexes with nucleic acid depends on the type of complex desired (surface charge, complex size and shape), the toxicity of the cationic polymer to the cell, and the environment (e.g., medium) in which the polymer is to be employed. The kinds and amounts of cationic polymer employed are typically balanced to minimize cell toxicity and maximize transfection efficiency. In preferred embodiments, the cationic polymer forms a complex with the nucleic acid that is to be transfected into cells. Preferably, nucleic acid complexes are formed by combining the nucleic acid with the cationic polymer prior to functional vesicle addition. Nucleic acid/cationic polymer complexes may then be encapsulated within functional vesicles via physical and chemical methods.

In preferred embodiments, transfection vectors include functional vesicles composed of a native or synthetic lipid bilayer and a biomolecule including viral envelope protein, or components thereof. The viral envelope protein may be wild-type, mutant, or genetically modified. In preferred embodiments, mutant or genetically modified envelope proteins retain the ability to enter eukaryotic cells. Some previous gene transfection techniques use whole virus in some form, including wild type virus, replication-deficient virus or virus inactivated by a variety of methods. Preferred embodiments avoid the safety risks and immune complications typical of these previous techniques by not utilizing whole virus in any form. In preferred embodiments, the production of viral envelope proteins requires only an envelope gene that has been isolated in some way. In some embodiments, the viral envelope gene is cloned into a mammalian gene expression plasmid. This plasmid, once transfected into cultured cells, will produce the viral protein. In preferred embodiments, in contrast to some previous techniques, there is no risk of regeneration of wild-type virus or of immunogenecity complications in a individual because the intact viral genome is not involved in the production of the envelope protein or the envelope vesicles, thus there are no viral proteins included in the envelope vesicles, other than molecules of the envelope protein.

Preferred embodiments use viral envelope components, especially viral envelope vesicles and viral envelope proteins, to enhance gene transfection efficiency and reduce cytotoxicity. The functional vesicles may include a native cell membrane or synthetic lipid bilayer, and may include membrane active polypeptides. Some examples of membrane active polypeptides include spike glycoproteins, multimers of spike glycoproteins (dimers, trimers or oligomers) and peptides of spike glycoproteins, any of which may function to enhance non-viral gene transfection into cells. Any proteins or peptides that have functions similar to viral envelope proteins described herein may be incorporated into lipid bilayer vesicles to encapsulate cationic polymer-nucleic acid complexes to achieve enhanced transfection and reduced cytotoxicity in particular embodiments.

In preferred embodiments, viral envelope components may be isolated by a variety of well-known techniques. The use of gradient ultracentrifugation for isolation of cellular components, as described in Abe, A. et al., In Vitro Cell-Free Conversion of Noninfectious Moloney Retrovirus Particles to an Infectious Form by the Addition of the Vesicular Stomatitis Virus Surrogate Envelope G Protein. J. Viology 1998, 72:6356-6361; and the use of spin filters and the cationic detergent DTAB for the isolation and purification of viral protein fractions, as described by Glushakova, et al., Isolation of influenza virus hemagglutinin and its separation into subunits by a stage-by-stage scheme for viral protein fractionation, Vopr Virusol. 1988, 33(3):286-9, are two known methods that can be adapted for the isolation of VSVG vesicles. Alternatively, membrane active polypeptides may be produced by a variety of standard genetic engineering synthesis methods. The peptide or biomolecule may be purified by an affinity column with the addition of tag or another ligand. In one variation of this method, the viral envelope gene within an expression plasmid is immediately preceded or followed by a genetic sequence that codes for a small peptide tag. The affinity column contains an antibody to the peptide tag and thus the column will bind the hybrid protein. The hybrid protein is eluted from the column, the tag is removed by enzymatic action and the viral envelope peptide is recovered. The viral functional peptides derived from envelope proteins also may be produced by standard chemical synthesis using automated solid phase peptide synthesis. It is apparent to those with ordinary skill in the art that a variety of methods may be used to generate membrane active polypeptides.

Media employed in transfection experiments done in accordance to some embodiments is similar to the medium used to culture cells for transfection. In preferred embodiments, media containing serum will have no significant effect on the efficiency of transfection. This simplifies the procedure of transfection for these embodiments and reduces the risk of contamination due to the extra steps of changing the transfection medium.

A variety of cationic polymers are known in the art. Examples of cationic polymers useful in this invention are listed in Table 1. Useful cationic polymers include those with a molecular weight over 400 Da, either linear or branched, biodegradable or non-biodegradable, with modification or without modification and with lipid conjugation or without lipid conjugation. Particular embodiments use polymers that are block copolymers or grafted copolymers.

It has been found that the following parameters may affect performance in a particular case: cationic polymer concentration, the molecular weight of cationic polymer, the concentration of nucleic acid, the methods of forming functional vesicles, the medium employed for transfection, the length of time the cells are incubated with transfection composition, the amount of functional vesicle or viral component employed, the ratio of each component in the complexes and the way in which the components of the transfection composition are combined into cationic polymer/DNA complexes. Routine experimentation, using the guidance provided herein, may be carried out to identify the proper parameter for a particular cell to be transfected.

It will also be apparent to those of ordinary skill in the art that methods, reagents, procedures and techniques other than those specifically detailed herein may be employed or readily adapted to produce the transfection vectors of the present invention and practice the transfection methods of this invention. Such alternative methods, reagents, procedures and techniques are within the spirit and scope of this invention.

The transfection compositions and methods of this invention are further illustrated in the following non-limiting Examples. All abbreviations used herein are standard abbreviations in the art. Specific procedures not described in detail in the Examples are well-known in the art.

### EXAMPLES

Cell Cultures and Plasmids

Standard tissue culture methods were employed. Human embryonic kidney transformed HEK 293T cells were maintained in Dulbecco's Modified Eagle Media (DMEM) (Gibco Inc.) containing 10% fetal bovine serum (FBS), 100 units/ml penicillin and 100 μg/ml streptomycin. In this media the cells had a doubling time of about 20 hours, and the cells were split every 3-4 days to avoid confluency.

The HeLa 705 cell line was derived from Human cervical carcinoma HeLa cells by introducing a firefly luciferase gene with a mutant β-globin intron (a mutation at 705 position) that expresses an inactive protein due to the incorrect splicing. However, the mutated intron can be corrected by a specific antisense oligonucleotide which blocks the mutant splicing site (Kang S H et al. Biochemistry 1998; 37(18):6235-9). The cell line was maintained in DMEM (Gibco) containing 10% fetal bovine serum, 100 units/ml penicillin and 100 μg/ml streptomycin. Two hundred μg/ml hygromycin was added into medium to maintain the luc-705 plasmid. In this media the cells had a doubling time of about 20 hours and were split every 3-4 days to avoid confluency.

Human liver tumor cell line HepG2 was maintained in A-MEM medium (Gibco, Inc.) containing 10% fetal bovine serum, 100 units/ml penicillin and 100 μg/ml streptomycin. In this media the cells had a doubling time of about 20 hours, and the cells were split every 3-4 days to avoid over confluency.

Human primary endothelial cell HUV-EC cell line was grew and maintained in EBM medium (Cambrex Corp.) containing 10% fetal bovine serum, 100 units/ml penicillin, 100 μg/ml streptomycin, and various growth factors as specified by the manufacturer.

Bovine artery endothelial cells (BAEC) and bovine aorta smooth muscle cells (BASMC) were isolated from bovine aorta and prepared as described according to known methods (Yu, L., Nielsen, M., and Kim, SW. TerpelxDNA Gene Carrier System Targeting to Artery Wall Cells. J. Controlled Release 72:179-189 (2001)). Briefly, aortas were taken from bovine cadavers at a slaughterhouse (Dale Smith and Sons, Draper, Utah). The endothelial cell (EC) cultures were prepared by lumenal digestion with 0.3% collagenase in PBS. The smooth muscle cell (SMC) cultures were prepared by dissection and enzymatic digestion with 0.3% collagenase and 0.4% elastase in DMEM. EC cells are supplemented with basic fibroblast growth factor (bFGF) for optimal growth and expression of normal cobblestone morphology. Cells used in transfection experiments were fed culture media (DMEM with 10% FBS) containing 20 ng bFGF/ml for at least five days prior to their use in a transfection assay. These cells were then trypsinized and plated out for transfection experiments. For a transfection experiment these cells were plated so that they formed an incomplete monolayer (70-80% confluence). SMC were cultured with DMEM medium containing 10% FBS. These cells were passaged in the same way as the ECs.

The plasmid pMNK-VSVG was constructed by cloning VSVG cDNA into plasmid pMNK with standard molecular methods. The expression of VSVG cDNA is controlled by human cytomegalovirus (CMV) promoter and the transcripts are stabilized by a gene expression enhancer, chicken β-globulin intron. The plasmid vectors pCMV-lacZ, pCMV-GFP and pCMV-luc were constructed by cloning the E. coli β-galactosidase gene, green fluorescent gene and firefly luciferase gene into pCMV-0, with the same backbone of mammalian expression vector, pMNK-VSVG, respectively. Plasmid DNA was amplified and purified with Qiagen EndoFree Plasmid Max Preparation Kid according to the manufacture's instruction.

Biodegradable Polymer Synthesis

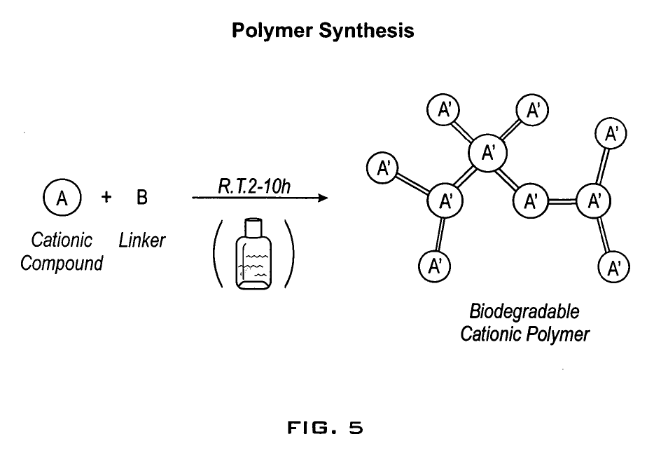

Synthesis of branched or slightly cross-linked biodegradable cationic polymers is illustrated in FIG. 5. This synthesis method can be used for preparation of large libraries of branched or slightly crosslinked biodegradable cationic polymers.

For example, in a preferred embodiment, A may represent an amine-containing cationic compound or oligomer with at least three reactive sites (for Michael addition reaction), and B may represent a compound having at least two acrylate groups (see FIG. 5). The polymerization reaction between A and B takes place under very mild conditions in organic solvents After the reaction, the polymers can be recovered by at least two different methods. In the first method, the polymers may be recovered by direct removal of the solvents at reduced pressure. In the second method, the polymers may be neutralized by adding acid, such as hydrochloric acid, and the neutralized polymers recovered by filtration or centrifugation. Branched or slightly cross-linked, water soluble polymers with high molecular weight can be obtained by controlling the ratio of A to B, reaction time, reaction temperature, solvents, and concentration of the solutes.

Polymers Prepared by Crosslinking Cationic Oligomers with Diacrylate Linkers, Recovered by Direct Removing Solvents

Synthesis of high molecular weight cationic polymers may be performed by a variety of methods known to those of ordinary skill in the art. The synthesis of a polymer which is derived from polyethylenimine oligomer with molecular weight of 600 (PEI-600) and 1,3-butanediol diacrylate (1,3-BDODA) is provided as a general procedure to serve as a model for other synthetic procedures involving similar compounds which can be used to synthesize other cationic polymers. 0.44 g of PEI-600 (Aldrich) was weighed and placed in a small vial, and 6 ml of methylene chloride was added. After the PEI-600 completely dissolved, 0.1 g of 1,3-BDODA in 2 ml of methylene chloride was added slowly into the PEI solution while stirring. The reaction mixture was stirred for 10 hours at room temperature. After removing the organic solvent under reduced pressure, 0.55 g of transparent, viscous liquid was obtained. ¹H-NMR spectrum indicated that the acrylic carbon-carbon double bond disappeared completely. The molecular weight of the obtained polymer was estimated by agarose gel electrophoresis. Several biodegradable cationic polymers (BCP-1, BCP-2, and BCP-3) were prepared in a similar manner and used in the transfection procedures described below, the results of which are shown in Tables 2-5, 7, and 8. Other branched or slightly crosslinked, degradable cationic polymers derived from other cationic oligomers and other linkers having structures similar to those used herein were prepared in a similar manner.

### Example 1

VSVG Vesicle Preparation by Biological Method

The plasmid pMNK-VSVG was transfected into 293 cells by superFect™ (Qiagen, Valencia Calif.) according to the manufacturer's instructions. Two days after transfection, VSVG vesicles were prepared by two methods. One method involved harvesting conditioned medium from 293 cells that were transfected with plasmid containing VSVG gene, pMNK-VSVG, filtering through (0.45μ) filter, followed by centrifugation at 30,000 rpm with a SW35 rotor for 60 min at 4° C. The pelleted VSVG vesicles were resuspended in phosphate-buffered saline (PBS) (pH 7.4), and the same volume of 60% sucrose PBS solution was added, followed by layering 4 ml of 20% and 10% of sucrose PBS solution and centrifuged at 30,000 rpm for 30 min at 4° C. The fractions containing VSVG vesicles were collected and dialysed against PBS for three changes for 20 hrs at 4° C.

The second method involved physically breaking the cell membrane, followed by sucrose gradient ultrancentrifugation separation. Briefly, pMNK-VSVG plasmids transfected 293 cells were treated with latex beads (polybead polystyrene microspheres 4.55×10 beads/ml, polysciences Inc.) for 1-2 hours prior to harvesting. After the bead treatment, the media was aspirated and the cells washed one time with 10 to 20 ml of buffer 1 (Ca⁺⁺ and Mg⁺⁺ free Phosphate Buffered Saline (PBS) solution, buffered with 0.02M Hepes pH=7.4). Cells were harvested by putting 20 ml of buffer 2 (Ca⁺⁺ and Mg⁺⁺ free PBS, buffered with 0.02 M Hepes, and 1 mM EDTA, pH=7.4) on each plate for 10 min in a 37° C. incubator. The cells were removed from plates by pipeting up and down, were transferred to a 50 ml tube and centrifuged at 4° C. at 150×g for 10 min.

All following steps were done at 4° C. The cell pellets were resuspended in 25 ml of PBS and the sample was transferred to tubes with 20 ml of 4% BSA PBS. The samples were centrifuged at 4° C. at 300 g for 15 min. The cell pellet was washed two times with PBS, followed by centrifugation at 4° C. at 150 g for 5 min for each wash. The cells were homogenized by a Dounce Homogenizer vigorously for 15 min. Five milliliters of 60% sucrose was added to the 5 ml of homogenate.

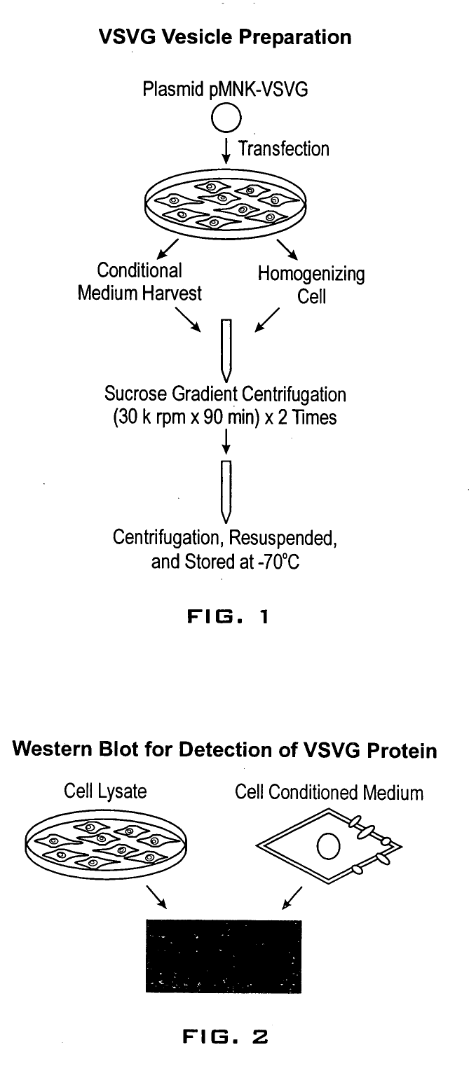

A volume of 3.330 ml of the homogenate-30% sucrose mixture was put into Beckman ultra clear centrifuge tubes and layered with 6 ml of 20% sucrose solution and 3 ml of 10% sucrose solution on the top. The samples were centrifuged in SW41 rotor buckets at 4° C. at 110,000 g (30,000 RPM) for 90 min. VSVG membrane fractions, found between the 10% and 20% sucrose layers, were collected and subjected to a second centrifugation in SW41 rotor buckets at 4° C. at 8000 RPM for 30 min. The VSVG vesicles were resuspended in 500 μl of 10% sucrose and stored at −70° C. The procedure for VSVG preparation is summarized in FIG. 1. FIG. 1 shows the general procedures of making VSVG vesicles from 293 cells (Human Embryonic Kidney Cell). The plasmid DNA carrying VSVG gene was tranfected into 293 cells and after 24 to 48 hours culture, the cells and medium were harvested separately through centrifugation. The pellets of VSVG vesicle were further separated from other components by gradient centrifugation methods. The prepared VSVG vesicle can be directly used in in vitro cationic polymer based transfection assays. VSVG concentration was determined by Coomassie plus protein assay kit (PIERCE, Rockford, Ill.) and VSVG was identified by immunobloting analysis, as seen in FIG. 2, according to standard molecular biology protocol. FIG. 2 shows the VSVG proteins purified from cell lysate or cell conditioned medium (supernatant) being identified by Western blot assay with an anti-VSVG antibody probe.

### Example 2

VSVG Vesicle Preparation by Synthetic Method

Chemicals: All Fmoc amino acids and rink amide MBHA resins were purchased from Nova Biochem. Dimethylformamide (DMF), piperidine, dichloromethane (DCM), 1-hydroxybenzotriazole (HOBt), 1,3-diisopropylcarbodiimide (DIC), N,N-diisopropylethylamine (DIPEA), diethyl ether, trifluoroacetic acid (TFA), triisopropylsilane (TIS), and acetic anhydride were obtained from Aldrich. Egg PC, brain PE, brain PS, and Triton X-100 were purchased from Sigma. PEI 1800 and PEI 25000 were supplied by Polysciences.

Peptide synthesis and conjugation: Peptides were synthesized by the standard F-moc (N-(9-fluorenyl)methoxycarbonyl) solid-phase method on the rink amide MBHA (4-Methylbenzhydrylamine HCl) resins. Briefly, the resins were swelling in DMF for 30 minutes. The Fmoc group was removed by treating the resins with 20% piperidine in DMF for 10 minutes. The resins were washed with DMF, DCM, and DMF, respectively. Then, amino acid previously dissolved in the mixture of DMF with HOBt was added to the resins. DIC and DIPEA were also added to the solution in order to form the OBt ester bond. Normally, the reaction was completed within 2 hours. Acetic anhydride and DIPEA were added to block any possibly uncompleted portions on the resin beads. The resins were subsequently washed with DMF, DCM, and diethyl ether. The whole process was repeated from the removal of Fmoc group until all amino acids were added. The peptide-resin conjugates were then cleaved with a mixture of TFA/water/TIS (95:2.5:2.5). The crude peptides were dried under vacuum overnight.

Solid-phase peptide conjugation was conducted by following the dimethyl sulfoxide-mediated oxidation method. Briefly, the peptides attached to the resins were treated with 1% TFA in DCM containing 5% TIS. The resins were washed with DMF, DCM, and diethyl ether. Additional crude peptides to be added to the peptide-resin were dissolved in 5% acetic acid in water and then added to the peptide-resin beads. The pH of solution was adjusted to 6 with ammonium carbonate and dimethyl sulfoxide was then added. The reaction was completed after 24 hours. The conjugated peptides were cleaved from the resins with a mixture of TFA/water/TIS (95:2.5:2.5). The conjugated peptides were then dried under vacuum overnight.

Vesicle formation: Vesicles were prepared from mixtures of egg PC/brain PE/brain PS at a ratio 6:2:2 (w/w/w). Peptides (peptide:lipid=1:100 mol/mol) previously dissolved in trifluoroethanol and cationic polymers (PEI 1800 or PEI 25000) previously dissolved in PBS were added to the egg PC/brain PE/brain PS mixture. GFP reporter gene was added to resulting mixture, followed by Triton X-100 (0.005% w/w). The solvent was evaporated under argon gas until a lipid film was obtained at the bottom of the flask. The film was then further dried under vacuum overnight. The lipid film was rehydrated in the buffer containing 10 mM Tris and 250 mM NaCl, pH 7.5, by shaking at 37° C. for 1 hour. The vesicle suspension was sonicated in a bath sonicator; the lipid suspension began to clarify and yielded a slightly hazy transparent solution within 40-60 minutes. During the sonication, the temperature in the water bath was maintained under 30° C. to prevent deterioration of liposomes. The lipid aggregates were then removed by centrifugation at 16,000 rpm for 20 minutes to yield a clear solution of small, unilamellar vesicles (SUVs). The resulting SUVs were used for gene transfection study in cell cultures.

Preparation of peptide vesicle-GFP reporter gene complexes: The peptide vesicle-GFP reporter gene complexes were freshly prepared before performing the experiment. Briefly, the plasmid containing GFP gene or antisense oligonucleotide with fluorescent dye tag (FITC) was added directly to the peptide SUVs. The mixture was incubated at room temperature for 10 minutes before use. Various amounts of Triton X-100 were added to the mixture to aid solubility. The peptide-lipid vesicle was constituted after dialysis against PBS pH 7.2.

Transfection of peptide vesicle-GFP reporter gene complexes in cell cultures: Cos-7 cells were seeded in 96-well plate the night before conducting the study to obtain the cell density about 60-70%. The medium was discarded and the cells were washed with PBS once before adding the flash prepared lipid-peptide vesicles with reporter plasmid, pCMV-GFP. After being incubated in 37° C. CO₂ incubator for 6 hours, the medium containing the vesicles were replaced by the Dulbecco's Modified Eagle Medium with 10% FBS. The cells were kept in 37° C. CO₂ incubator for 24 hours before observing the signal under the microscope.

### Example 3

Transfection Assays in 293 and HT1080 Cells by Adding VSVG Vesicles

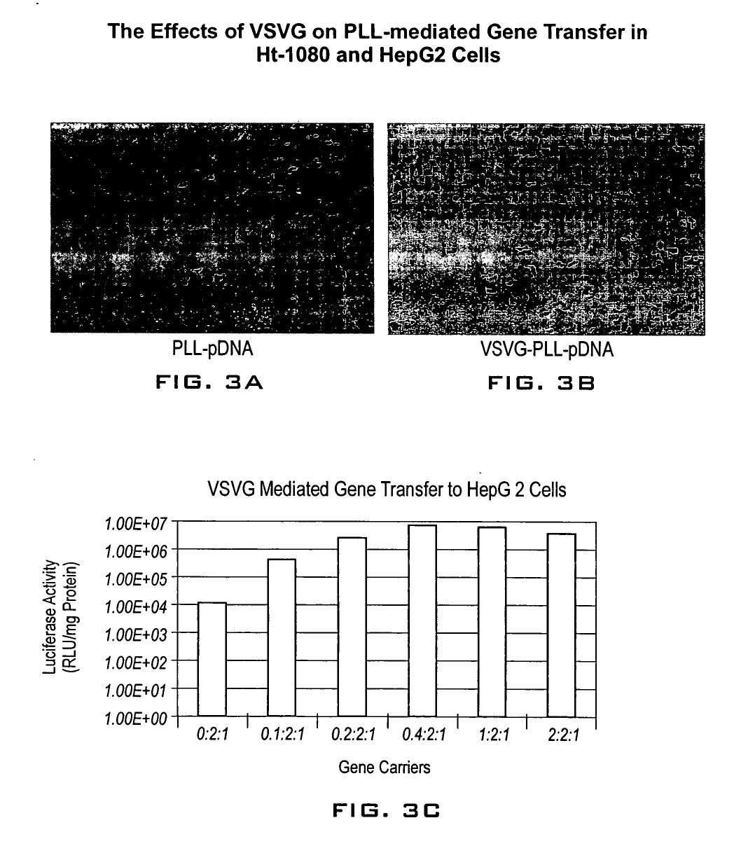

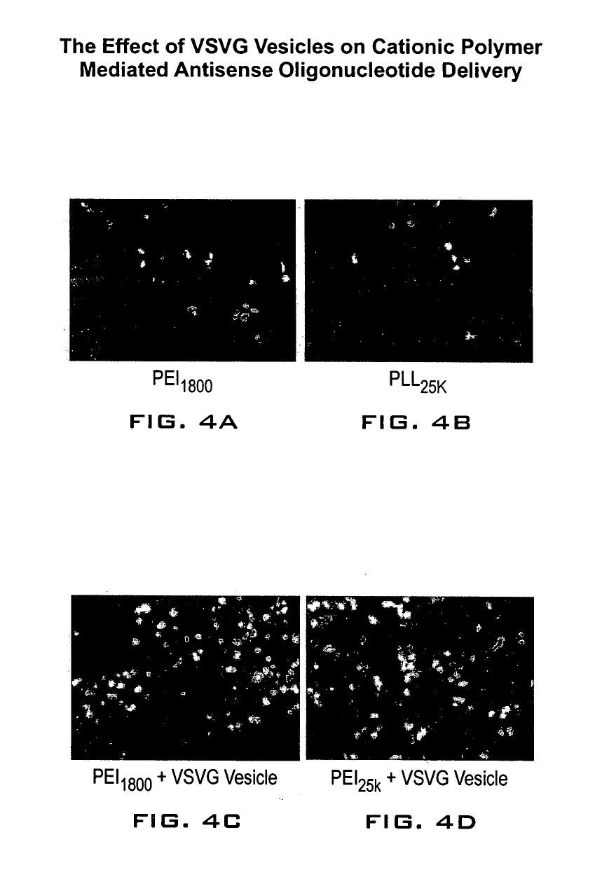

PLL, PEI, polyamidoamine (PAMAM, dendrimer) and biodegradable polymers were used for transfection of plasmid DNA into mammalian cells in vitro to evaluate the effect of VSVG vesicles on cationic polymer mediated gene transfer, as is illustrated in FIG. 3. The cultured cells (from cell lines 293 and HT1080) were plated in 24-well tissue culture plates (1×10⁵ cells/well for 293 cells and 5×10⁴ cells/well for HT1080) and incubated overnight in DMEM with 10% FBS. The primary cells (bovine aorta endothelial cells (6×10⁴ cells/well) and bovine aorta smooth muscle cells and 3×10⁴ cells/well) were plated in 24-well tissue culture plates and incubated overnight in DMEM with 10% FBS and 20 ng bFGF/ml. For each well, an aliquot of 100-μl DNA solution containing 1 μg of plasmid DNA, e.g. pCMV-lacZ plasmid DNA or pCMV-luc, was mixed with 100-μl cationic polymer solution containing 2 μg of PLL or 0.25 μg of PEI. The DNA and cationic polymer solutions were mixed and incubated for 10-15 min at room temperature to allow the formation of DNA-cationic polymer complexes. Various amounts of VSVG vesicles in 100 μl of 10% glucose solution were added to the DNA-cationic polymer complex-containing solutions and were then added to the cells in individual wells after the cells were washed with PBS. Cells were incubated (37° C., 5% CO²) for 24 hrs without changing the medium, after which they were assayed for E. coli beta-galactosidase and fruitfly luciferase activities using the methods described below.

β-galactosidase Activity Cytochemical Assay

In situ staining was used to demonstrate E. coli beta-galactosidase gene expression as a standard procedure. Cells were rinsed with PBS, fixed for 5 min in 2% (v/v) formaldehyde, 0.2% glutaraldehyde in PBS, rinsed twice with PBS, and stained 2 hours to overnight with 0.1% X-gal, 5 mM potassium ferricyanide, 5 mM potassium ferrocyanide, 2 mM MgCl₂ in PBS. Rinsed cells were photographed using a 10× objective on a Olympus inverted microscope. The percent of stained cells in transfected cultures was determined from counts of three fields for optimal cationic polymer amounts. The results of transfections of 293, HT1080 and bovine artery wall primary cells with pCMV-lacZ using various transfection reagents are presented in FIG. 3. FIG. 3 shows that the effects of VSVG vesicles on cationic polymer-mediated gene delivery in HepG2 cells (hepatic carcinoma cells) via staining for β-galactosidase with X-gal (for visualization) and via luciferase activity measurement (quantification). With the VSVG vesicles, the number of β-galactosidase-positive cells transfected by cationic polymer (poly-L-lysine, PLL) (FIG. 3B) increased to 20 to 25%, compared to the less than 5% positive cells for transfection with PLL alone (FIG. 3A). Using a luciferase reporter gene assay, the VSVG vesicles were shown to enhance PLL-mediated gene transfection efficiency from 10⁴ to 10⁷, three orders higher, as compared to PPL-mediated transfection without VSVG vesicles (FIG. 3C). Ratios given in FIG. 3C are vesicle:PLL:plasmid DNA.

GFP Reporter Gene Transfection Assay

Green fluorescent protein (GFP) gene was used in an initial screening. After transfection, the GFP signal in cells was observed under fluorescent microscope (Olympus, filter 520 nm). Cells were photographed using a 10× objective. The percentage of cells with GFP signal in transfected cultures was determined from counts of three fields for optimal cationic polymer amounts. The results of experiments done to study the effects of VSVG vesicles on the transfection of GFP constructs are found in Tables 3 and 4 below.

Luciferase Activity Assay

Measurement of luciferase activity was performed using a chemiluminescent assay following the manufacturer's instructions (Luciferase Assay System; Promega, Madison, Wis., USA). Briefly, thirty hours after gene transfer, the cells were rinsed twice with PBS and then were lysed with lysis buffer (1% Triton X-100, 100 mM K₃PO₄, 2 mM dithiothreitol, 10% glycerol, and 2 mM EDTA pH 7.8) for 15 min at room temperature. A 20-μl aliquot of cell lysate was then mixed with 50-μl of luciferase assay reagent with injector at room temperature in the luminometer. Light emission was measured in triplicate over 10 seconds and expressed as RLUs (relative light units). Relative light units (RLU) were normalized to the protein content of each sample, determined by BCA protein assay (Pierce, Rockford, Ill.). All the experiments were conducted in triplicate. The results of transfection of 293, HT1080 and bovine artery wall primary cells with pCMV-luc using various transfection reagents are presented in Tables 1 and 2.

TABLE 1
The effect of VSVG Vesicles on Cationic Polymer Mediated
Gene Transfer
Transfection Efficiency (RLU/mg of protein)

| | | |
|---|---|---|
| Polymers | Without VSVG | With VSVG |

In Vitro in 293 cells

| | | |
|---|---|---|
| PLL | 25000 +/− 12000 | 13000000 +/− 6500000 |
| PEI | 670000 +/− 140000 | 45000000 +/− 3400000 |
| Dendrimer | 950000 +/− 230000 | 56000000 +/− 7600000 |

HT1080 cells

| | | |
|---|---|---|
| PLL | 15000 +/− 16000 | 11000000 +/− 3500000 |
| PEI | 330000 +/− 90000 | 23000000 +/− 800000 |
| Dendrimer | 760000 +/− 270000 | 41000000 +/− 3800000 |

Bovine Endothelial Cells

| | | |
|---|---|---|
| PLL | 160 +/− 80 | 32000 +/− 13000 |
| PEI | 9200 +/− 7800 | 66000 +/− 35000 |
| Dendrimer | 11000 +/− 8700 | 72000 +/− 54000 |

Bovine Smooth Muscle Cells

| | | |
|---|---|---|
| PLL | 120 +/− 98 | 31000 +/− 24000 |
| PEI | 8500/7600 | 112000 +/− 65000 |
| Dendrimer | 21300 +/− 15000 | 106000 +/− 35000 |

The data in Table 1 show the effect of VSVG vesicles on enhancing cationic polymer mediated gene transfer in various cell lines including transformed cells (293 cells and HT1080 cells) and primary cells (bovine endothelial cells and bovine smooth muscle cells). The transfection efficiencies were measured by luciferase activities. Three cationic polymers were selected in these studies, including linear cationic polymer with only primary amine groups (PLL), the branched cationic polymer with primary, secondary, and tertiary amine groups (PEI), and cationic dendrimer. With VSVG vesicle help, the cationic polymer mediated transfection efficiencies were at least 100 times higher compared to that with cationic polymer alone.

TABLE 2
The Effect of VSVG Vesicles On Degradable Polymer Mediated
Gene Delivery
Transfection Efficiency (RLU/mg of protein)
In Vitro in 293 cells

| Polymers | Without VSVG | With VSVG |
|---|---|---|
| BCP-1 | 2,500,000 +/− 120,000 | 27,000,000 +/− 6,500,000 |
| BCP-2 | 6,700,000 +/− 140,000 | 72,000,000 +/− 340,000 |
| BCP-3 | 9,500,000 +/− 230,000 | 110,000,000 +/− 7,600,000 |

The data in Table 2 show that VSVG vesicles also enhanced biodegradable cationic polymer mediated transfection efficiencies significantly (at least 10 times higher) in 293 cells with luciferase reporter gene.

TABLE 3
The Effect of Synthetic VSVG Vesicles On Cationic Polymer Mediated
Reporter Gene GFP Delivery
Transfection Efficiency (GFP Positive Cells, %)
In Vitro in COS7 cells

| | Without Synthetic Vesicle | With Synthetic Vesicle |
|---|---|---|
| Polymers | | |
| PLL₂₅ₖ | 3% +/− 1% | 19% +/− 6% |
| PEI₁₈₀₀ | 11% +/− 3% | 23% +/− 8% |
| Biodegradable | | |
| BCP-1 | 35% +/− 4% | 46% +/− 3% |
| BCP-2 | 37% +/− 7% | 51% +/− 5% |

The data in Table 3 show that synthetic VSVG vesicles also enhanced cationic polymer mediated gene transfer efficiencies in COS7 cells with green fluorescent protein reporter gene. Using synthetic VSVG vesicles, the number of GFP positive cells increased by 6 fold as compared to those transfected without using VSVG vesicles.

TABLE 4
The Effect of Synthetic VSVG Vesicles On Cationic Polymer
Mediated FITC-Antisense Oligonucleotide Delivery
Transfection Efficiency (FITC Positive Cells, %)
In Vitro in COS7 cells

| Polymers | Without Synthetic Vesicle | With Synthetic Vesicle |
|---|---|---|
| PLL₂₅ₖ | 1% +/− 0.5% | 16% +/− 6% |
| PEI₁₈₀₀ | 7% +/− 3% | 28% +/− 5.5% |
| BCP-1 | 23% +/− 5% | 35% +/− 7% |
| BCP-2 | 21% +/− 5% | 32% +/− 4% |

The data in Table 4 demonstrate the effects of synthetic VSVG vesicles on cationic polymer (biodegradable and non-biodegradable) mediated GFP reporter gene delivery efficiencies. With the use of synthetic VSVG vesicles, the efficiency of cationic polymer-mediated GFP reporter gene transfer increased by up to 10 fold as compared to the equivalent transfections done without synthetic VSVG vesicles.

TABLE 5
The Effect of VSVG Vesicles On Cationic Polymer Mediated siRNA
Delivery Transfection Efficiency (GFP Positive Cells, %)
In Vitro in 293 cells

| Polymers | Without VSVG Vesicle | With VSVG Vesicle |
|---|---|---|
| PLL₂₅ₖ | 85% +/− 7% | 65% +/− 8% |
| PEI₁₈₀₀ | 89% +/− 6% | 67% +/− 9% |
| BCP-3 | 45% +/− 6% | 17% +/− 9% |

The data in Table 5 show the effect of VSVG vesicles on cationic polymer mediated siRNA delivery, evaluated by percentage of GFP positive cells. The vesicles are used to transfect cells that constitutively express GFP protein. The successful delivery of siRNA to a cell results in the inhibition of GFP expression in that cell. In all transfections, the GFP gene expression was inhibited to a degree after the addition of the complex of cationic polymer and siRNA. Lower percentages of GFP positive cells indicates better inhibition of GFP expression, which in turn indicates a higher efficiency in siRNA delivery. The data in table 8 indicated that VSVG vesicles significantly enhance siRNA delivery compared to delivery of siRNA by cationic polymer alone.

### Example 4

Cytotoxicity Assays in HT1080 Cells Adding VSVG Vesicle

PEI and polyamidoamine dendrimer were selected for evaluation of the effects of viral envelope vesicles on the reduction of cationic polymer gene carrier cytotoxicity, because PEI and dendrimer were reported to have a high toxicity to the cells both in vitro and in vivo studies. The cytotoxicities of cationic gene carrier on mammalian cells were evaluated using 3-[4,5 dimethylthiazol-2-yl]-2,5-diphenyltetrazolium bromide (MTT) described by Yu et al., (Yu, L., Nielsen, M., and Kim, S W. TerpelxDNA Gene Carrier System Targeting to Artery Wall Cells. J. Controlled Release 72:179-189 (2001)). Briefly, HT1080 cells, 2×10⁴ cells/well, were seeded in 96-well plates and incubated for 24 hr. Various amounts of polymeric carriers were added to the cells for a period of six hours. The media was then removed and fresh media added. Following further incubation for 48 hrs, the media was removed and 200-μl of MTT solution (0.5 mg/ml) was added to each well, and incubated for 3 hrs. The medium was then removed and 200-μl DMSO was added to dissolve the formazan crystals. The absorbance of the solution was measured at 570 nm. Cell viabilities was calculated using the equation: Viability (%)={Abs_{570 (sample)}/Abs_{570 (control)}}×100. All the experiments were conducted in triplicate. Results obtained with optimal PEI amounts were compared in Table 6. Briefly, HT1080 cells, 2×10⁴ cells/well, were seeded in 96-well plates and incubated for 24 hr. Various amounts of VSVG vesicles were added to PEI/pDNA complex (500 ng/500 ng) solutions and then were added to the cells for a period of six hours. The remaining steps are the same as those used in the cytotoxicity assay described above. All the experiments were conducted in triplicate. Results obtained with VSVG vesicles and synthetic VSVG vesicles with cationic polymer were showed in Table 7.

TABLE 6
The Effect of VSVG Vesicles on Reducing Cytotoxicity of PEI and
Polyamidoamine Dendrimer

Cell Viability

| VSVG amount | PET/DNA (1:1) | Dendrimer/DNA (25:1) |
|---|---|---|
| 0 | 0 | 0 |
| 12.5 | 35% +/− 5% | 37% +/− 4% |
| 25 | 44% +/− 7% | 46% +/− 6% |
| 50 | 65% +/− 9% | 68% +/− 10% |
| 100 | 74% +/− 13% | 76% +/− 7% |
| 200 | 81% +/− 8% | 83% +/− 12% |
| 500 | 85% +/− 11% | 89% +/− 8% |

The data in Table 6 show that VSVG vesicles also reduced cationic polymer-induced cytotoxicity and improved cell viabilities. The data also shows that the protective effect of the VSVG vesicles is dose dependent and, in these particular experiments, reached a saturated state when 500 ng of VSVG protein was used.

TABLE 7
The Effect of VSVG Vesicles and Synthetic VSVG Vesicles on Reducing
Cytotoxicities Of Cationic Polymer/Plasmid DNAComplex and Cationic
Polymer/Antisense Oligonucleotide Complex
Cell Viability (MTT Assay)
In HT1080 Cells

| Polymer-DNA | | |
|---|---|---|
| Complexes | Without VSVG Vesicle | With VSVG Vesicle |
| PLL₂₅ₖ-pCMV-GFP | 45% +/− 4% | 92% +/− 6% |
| BCP-1-pCMV-GFP | 75% +/− 3 | 97% +/− 2 |

In HT1080 Cells

| Polymer-DNA | | |
|---|---|---|
| Complexes | Without Synthetic Vesicle | With Synthetic Vesicle |
| PLL₂₅ₖ-pCMV-GFP | 45% +/− 4% | 89% +/− 8% |
| BCP-1-pCMV-GFP | 75% +/− 3 | 91% +/− 4% |

The data in Table 7 show that both native and synthetic VSVG vesicles protected cells from cationic polymer-induced damage. The percentage of cells that are viable after transfection can double with the use of VSVG vesicles.

### Example 5

Use of Mellitin as a Membrane Active Polypeptide with Lipid-Based Vesicles for Transfection

Mellitin is the major component of bee venom. It is composed of 26 amino acids and forms a cationic peptide that disrupts membranes. In aqueous solution, melittin forms amphipathic α-helices that interact with lipid membranes via a positively charged cluster (KRKR) near the C terminus, inserting into the lipid bilayer and perturbing the structure. These activities, combined with its net positive charge, make melittin an interesting candidate for enhancing the delivery of DNA in transfection protocols. In these experiments, synthetic mellitin vesicles were prepared in the same manner as the synthetic VSVG vesicles above. The synthetic mellitin vesicles can significantly enhance the efficiency of gene transfection mediated by biodegradable polymer (BCP-3) and non-biodegradable (PLI₂₅ₖ and PEI₁₈₀₀) cationic polymers by up to 15 fold.

TABLE 8
The Effect Of Mellitin Peptide Vesicle On Enhancing Cationic
Polymer Mediated Gene Delivery.
Transfection Efficiency (GFP Positive Cells %)
In Vitro in COS7 cells

| Polymers | Without Synthetic Vesicle | With Synthetic Vesicle |
|---|---|---|
| PLL₂₅ₖ | 1.3% +/− 0.6% | 18% +/− 5% |
| PEI₁₈₀₀ | 5.7% +/− 4% | 31% +/− 4% |
| BCP-3 | 23.7% +/− 6.5% | 41% +/− 5% |

The data in Table 8 show the effects of the vesicles on the efficiency of transfection. The synthesized mellitin-vesicles significantly enhanced cationic polymer-mediated gene delivery to mammalian cells, up to 14 fold in case of poly-L-Lysine-mediated GFP reporter gene delivery.

1. A vector for transfecting a eukaryotic cell, comprising a nucleic acid, a biodegradable cationic polymer, a lipid-based vesicle and a viral envelop protein.

2. The vector of claim 1, having a transfection efficiency and cytotoxicity that is improved in relation to a comparable vector comprising said polymer in the absence of said lipid-based vesicle and said viral envelop protein.

3. The vector of claim 2, wherein said nucleic acid is selected from the group consisting of DNA, RNA, and DNA/RNA hybrid.

4. The vector of claim 3, wherein said DNA is selected from the group consisting of a linear molecule, a circular molecule, and a single stranded oligodeoxynucleotide.

5. The vector of claim 4, wherein said circular molecule is plasmid DNA.

6. The vector of claim 3, wherein said RNA is selected from the group consisting of single stranded RNA and double stranded RNA.

7. The vector of claim 6, wherein said single stranded RNA is a ribozyme.

8. The vector of claim 6, wherein said double stranded RNA is a small interference RNA.

9. The vector of claim 1, wherein said biodegradable cationic polymer has a molecular weight of at least 400 Da.

10. The vector of claim 1, wherein the molecular structure of said biodegradable cationic polymer is selected from the group consisting of linear, branched, dendrimer and star-shaped.

11. The vector of claim 1, wherein said nucleic acid biodegradable cationic polymer is a graft copolymer or a block copolymer.

12-14. (canceled)

15. The vector of claim 121, wherein said biodegradable cationic polymer is selected from the group consisting of a hydrolysable polymer, a pH sensitive cleavable polymer, a light sensitive cleavable polymer, a temperature sensitive cleavable polymer, a sonication sensitive cleavable polymer, and an enzymatically cleavable polymer.

16. The vector of claim 441, wherein said biodegradable cationic polymer comprises linkers and oligomers, the oligomers being selected from the group consisting of poly-L-lysine, polyethylenimine, poly[a-(-aminobutyl)-L-glycolic acid], chitosan, polyamidoamine, and poly(2-dimethylamino)ethyl methacrylate.

17. The vector of claim 10, wherein said dendrimer has more than three branches.

18. The vector of claim 441, wherein said biodegradable cationic polymer and said nucleic acid are present in a weight ratio in the range of about 1:1 to 50:1.

19. The vector of claim 1, wherein said lipid-based vesicle comprises a material selected from the group consisting of a mammalian cell membrane and a lipid mixture.

20. The vector of claim 19, wherein said lipid mixture comprises phosphatidylcholine, phosphatidylethanolamine and phosphatidylserine.

21. The vector of claim 20, wherein said phosphatidylcholine, said phosphatidylethanolamine and said phosphatidylserine are present in a ratio of about 6:2:2 by weight, respectively.

22-23. (canceled)

24. The vector of claim 1, wherein said viral envelop protein is selected from the group consisting of a wild-type envelope protein and a recombinant envelope protein.

25. The vector of claim 1, wherein said viral envelop protein is a vesicular stomatitus virus glycoprotein.

26. The vector of claim 1, wherein said viral envelop protein comprises a monomer of vesicular stomatitus virus glycoprotein.

27. The vector of claim 25, wherein said vesicular stomatitus virus glycoprotein is selected from the group consisting of a wild type vesicular stomatitus virus glycoprotein mature protein, a wild type vesicular stomatitus virus glycoprotein peptide and a recombinant vesicular stomatitus virus glycoprotein polypeptide.

28. The vector of claim 1, wherein said biodegradable cationic polymer and said nucleic acid are in the form of a complex.

29. The vector of claim 28, wherein said complex is contained within said lipid-based vesicle.

30. The vector of claim 28, wherein said complex is in contact with said lipid-based vesicle.

31. A method of making the vector of claim 1, comprising:
combining a nucleic acid with a biodegradable cationic polymer to form a complex;
providing a plurality of lipid-based vesicles, said lipid-based vesicles comprising at least one viral envelop protein; and
combining said complex with said plurality of lipid-based vesicles.

32. (canceled)

33. The method of claim 31, wherein the biodegradable cationic polymer comprises linkers and oligomers, the oligomers being selected from the group consisting of poly-L-lysine, polyethylenimine, poly[a-(-aminobutyl)-L-glycolic acid], chitosan, polyamidoamine, and poly(2-dimethylamino)ethyl methacrylate.

34. (canceled)

35. The method of claim 3431, wherein said biodegradable cationic polymer is selected from the group of consisting of a hydrolysable polymer and a pH sensitive cleavable polymer.

36. (canceled)

37. The method of claim 35, wherein said pH sensitive cleavable polymer is a polyacetal polymer.

38. The method of claim 31, wherein said lipid-based vesicles comprise a native lipid membrane.

39. The method of claim 31, wherein said lipid-based vesicles comprise a synthetic lipid membrane.

40. The method of claim 31, wherein said viral envelop protein is selected from the group consisting of vesicular stomatitus virus glycoprotein and a portion of vesicular stomatitus virus glycoprotein that is membrane active.

41. A method of gene therapy comprising:
identifying an individual in need of gene therapy; and
administering the vector of claim 1 to said individual in a therapeutically effective amount.

42. The method of claim 41, wherein said individual is a mammal.

43. A method of introducing a nucleic acid into a cell comprising contacting said cell with the vector of claim 1.

44. The method of claim 43, wherein said cell is a eukaryotic cell selected from the group consisting of a human fibroblast, an animal embryo stem cell, a keratinocyte, a pancreatic cell, a myocardium cell, a bone marrow cell, a neuronal cell, and a macrophage.
